(19) United States
(12) Patent Application Publication
(10) Pub. No.: US 2024/0002630 A1
(43) Pub. Date: Jan. 4, 2024

(54) XRF-IDENTIFIABLE BLACK POLYMERS

(71) Applicant: SECURITY MATTERS LTD., D.N. Hevel Eilot (IL)

(72) Inventors: Haggai ALON, Kibbutz Naan (IL); Tehila NACHUM, Holon (IL); Mor KAPLINSKY, Herzelia (IL); Ron DAFNI, Tel Aviv (IL); Nataly TAL, Nes Ziona (IL); Chen NACHMIAS, Tel Aviv (IL); Hagit SADE, Ramat Gan (IL); Gal SHMUELI, Or Yehuda (IL); Yonatan MUSNIKOW, D.N. Hevel Eilot (IL); Nadav YORAN, Tel Aviv (IL)

(21) Appl. No.: 18/255,496

(22) PCT Filed: Dec. 2, 2021
(86) PCT No.: PCT/IL2021/051437

§ 371 Date: Jun. 1, 2023

Related U.S. Application Data
(60) Provisional application No. 63/121,066, filed on Dec. 3, 2020.

(30) Foreign Application Priority Data
Apr. 21, 2021 (IL) 282500

(51) Int. Cl. C08K 3/04; C08K 3/30; C08K 3/08; C08K 3/28; C08K 3/22; C08J 3/22; B07C 5/346; G01N 23/223; G01N 33/44

(52) U.S. Cl. CPC C08K 3/04; C08K 3/30; C08K 3/08; C08K 3/28; C08K 3/22; C08J 3/226; B07C 5/346; G01N 23/223; G01N 33/442; C08K 2201/014; C08K 2003/3009; C08K 2003/0806; C08K 2003/282; C08K 2003/2251; C08K 2003/2262; C08K 2201/011; C08J 2323/06; B07C 2501/0054; G01N 2223/0766

(57) ABSTRACT

The invention subject of the present application concerns sorting of black plastics.

### TECHNOLOGICAL FIELD

The invention generally concerns black polymers and methods of marking the same.

### BACKGROUND

Carbon black is one of the most common additives in the polymer industry. It is widely used in preparation of black plastic for a variety of fields and industries. Among the common uses are building and construction, healthcare, packaging, houseware, electronics and appliances, as well as in the automotive and aircraft industries.

Despite the wide use of black plastic, most of it is not recyclable. This is mainly due to the fact that black plastic is not identifiable by common optical sorting systems used in recycling plants. Hence, products made from black plastic usually end up reaching the end of the processing line as waste.

International Patent Application Publication No. WO2018/069917 describes formulations and masterbatches of a polymeric material and XRF-identifiable markers, for producing transparent elements comprising a polymer and at least one XRF-identifiable marker for a variety of industrial uses.

### BACKGROUND ART

[1] WO2018/069917

### GENERAL DESCRIPTION

Health concerns surrounding use of black plastic stem mainly from the fact that it is almost never recycled. The material is not easy to recycle because it gets its color from carbon black, a type of industrial pigment additive used for its durability and deep shade. The black pigment is not easily identified by infrared sensors typically used in most plastic sorting facilities to separate out different types of plastic materials. This means that these plastics and their chemical additives end up in landfills or on the side of the road. The toxic chemicals can then find their way into the environment and end up in drinking water.

Coupled with the fact that despite the broad use seen in recent years for black plastic, uncolored plastic makes most of the plastic products available. Thus, little incentive has been expressed to create better sorting technology to address the increasing use of black plastic and the consequent health hazards it creates. Great efforts which have been invested in replacing carbon black with other black pigments that do not absorb IR have failed as carbon black was found superior to other black.

The inventors of the technology disclosed herein have developed a unique methodology that enables simple, cost effective and facile detection of black plastics, thus permitting efficient sorting thereof. The methodology of the invention concerns uses of a novel carbon black formulation which comprises in addition to carbon black at least one XRF-identifiable material. Without altering the mechanical and chemical properties of the carbon black, in formulating a novel formulation of the invention, an amount of an XRF-identifiable material is added into carbon black and mixed to form a novel pigment or reinforcing material that can be implemented in a variety of products for tracing, authentication of generally identifying the history of the product.

As known in the art, “carbon black” is a fine particulate matter, typically composed of ultrafine particles having diameters smaller than 2.5 μm and typically in the nanometric range. Carbon black typically contains pure carbon with a high surface-area-to-volume ratio. As a pigment, carbon black is widely used in various applications from black coloring pigment of newspaper inks to electric conductive agents of high-technology materials. The material is also used as a reinforcing agent for increasing the strength, particularly the abrasion resistance and tear strength of polymeric compositions or composites comprising same.

Carbon Black is the most widely used and cost-effective rubber reinforcing agent in tire components (such as treads, sidewalls and inner liners), in mechanical rubber goods, including industrial rubber goods, membrane roofing, automotive rubber parts (such as sealing systems, hoses and anti-vibration parts) and in general rubber goods (such as hoses, belts, gaskets and seals).

Despite similar names, carbon black should not be confused with black carbon, which is excluded from aspects of the invention.

Thus, in a first aspect of the invention, there is provided a composition comprising carbon black and at least one XRF-identifiable material, the composition being (for use as) a pigment formulation or a reinforcement formulation, wherein the at least one XRF-identifiable material is present in an amount selected to provide an XRF-identifiable signature indicative of the carbon black or the composition comprising same.

Similarly provided is a composition consisting carbon black and at least one XRF-identifiable material, the composition being (for use as) a pigment formulation or a reinforcement formulation, wherein the at least one XRF-identifiable material is present in an amount selected to provide an XRF-identifiable signature indicative of the carbon black or the composition comprising same.

The amount of the XRF-identifiable material added to or present in a composition or a product of the invention, or the amount that is used for the purpose of identifying and sorting a black object containing the marker, is a predetermined amount that provides a signature defining a material characteristics or attributes or profile. Thus, an amount of a salt or a material that may be regraded XRF-identifiable, but which may be present in a composition or other products of the invention for modulating other properties of the material, and thus not preselected and added in accordance with the invention, does not provide a signature on the basis of which the composition or product made therefrom can be identified or read. In other words, presence of an amount of an XRF-identifiable material that is not added in accordance with the invention to define a signature indicative of the composition or product, is not regraded falling within the scope of the present invention.

In some embodiments, the amount of the XRF-identifiable marker in the composition is between 50 and 300 ppm. In some embodiments, the amount is between 50 and 70 ppm, 50 and 100 ppm, 50 and 150 ppm, 50 and 200 ppm, 50 and 250 ppm, 70 and 100 ppm, 70 and 150 ppm, 70 and 200 ppm, 70 and 250 ppm, 70 and 300 ppm, 100 and 150 ppm, 100 and 200 ppm, 100 and 250 ppm or between 100 and 300 ppm. In other words, the amount is between 50 and 60 ppm, 50 and 70 ppm, 50 and 80 ppm, 50 and 90 ppm or 50 and 100 ppm.

In some embodiments, the composition comprises or consists the carbon black, the XRF-identifiable material and a polymer or a prepolymer, as defined.

In some embodiments, the composition is in a form of a solid composition, a dispersion, or a liquid composition comprising the components disclosed herein in dispersion, suspension or solubilized form(s).

In some embodiments, the composition of the invention is in a form of a concentrate that may be diluted by adding an amount thereof into a polymeric material or mixture from which black objects may be formed. The amount of the XRF-identifiable material in such objects to be formed from the composition provide an XRF-identifiable signature indicative of the product profile, namely one or more of date of manufacture, site of manufacture, composition, presence or absence of unnatural additives, and others. Where the product is a recycled product, namely of a polymer or polymeric composition that has been previously made and used, the profile may include data relating to such prior uses.

Also provided is a pigment formulation comprising carbon black and an amount of at least one XRF-identifiable material.

Also provided is a pigment formulation comprising carbon black and an amount of at least one XRF-identifiable material, wherein the amount of the XRF-identifiable material defining an electromagnetic radiation signature indicative of the material composition of the pigment formulation or the product to be marked therewith and/or production profile of the product (e.g., the raw material data). The profile may include one or more date of manufacture, site of manufacture, composition, presence or absence of unnatural additives, etc.

In some embodiments, the pigment formulation is provided as a powder or pellet form, wherein the amount of the at least one XRF-identifiable material is selected to provide an XRF marked product having an identifiable and XRF signature.

Also provided is a reinforcing agent, e.g., for improving at least one mechanical property of a polymer or a polymeric composite, the agent comprising carbon black and at least one XRF-identifiable material. In some embodiments, the agent is provided as a powder or pellet form, wherein the amount of the at least one XRF-identifiable material is selected to provide an XRF marked product having an identifiable and XRF signature.

Further provided is a pelletized powder comprising a homogenous blend of carbon black and at least one XRF identifiable marker.

In some embodiments of formulations of the invention, the pigment or reinforcing formulation may be presented as a solid powder formulation or combination of solid materials or in a liquid suspension or dispersion form. In some embodiments, such formulations may also comprise a polymer or a prepolymer.

Thus, in accordance with additional aspects, the present invention provides an XRF-identifiable masterbatch comprising a homogenous blend of carbon black, at least one XRF identifiable marker and at least one polymer or prepolymer. In some embodiments, the polymer is a thermoplastic polymer or a thermoset polymer. In some embodiments, and as further defined hereinbelow, the polymer may be selected specifically from Low-Density Polyethylene (LDPE), Linear Low-Density Polyethylene (LLDPE), High-Density Polyethylene (HDPE), Polypropylene (PP), Polyisoprenes, natural rubber and latex.

In accordance with some further aspects, the present disclosure provides an article of manufacture formed from or comprising a formulation of the invention, namely comprising carbon black, at least one XRF identifiable marker and at least one polymer, e.g., thermoplastic polymer.

In accordance with yet some other aspects, the present disclosure provides a method of preparing an XRF-identifiable article of manufacture, the method comprising:
(i) pelletizing a mixture comprising carbon black and at least one XRF-identifiable marker;
(ii) melt blending pellets obtained from said pelletizing with at least one thermoplastic polymer to form a molten mixture;
(iii) molding the molten mixture to obtain said article of manufacture.

As noted herein, carbon black is used to strengthen rubber and other polymers, and also acts as a pigment, UV stabilizer, and conductive or insulating agent in a variety of rubber, plastic, ink and coating applications. Apart from tires to which carbon black gives their color, carbon black is also used in garden hoses, conveyor belts, plastics, printing inks and automotive coatings. Thus, articles of manufacture that are within the scope of the invention include tires, plastic products, printed products (2D or 3D products), and others.

As stated throughout the present disclosure, the inability to sort black plastic or other black polymers in which carbon black is used raises the need for a novel approach for proper marking of raw materials and for managing the recycling and reuse of various materials comprising such black raw materials, in particular black plastic materials, by timely performing decision making and generating corresponding sorting data for each black plastic material and preferably also generating a corresponding certificate assigned to said black plastic material. Such sorting data, generated based on real time inspection of the properties/conditions of the black raw material as well as of each black plastic material, is indicative of whether successive recycling of said black plastic material allows its further use in a product, and the suitable product type.

As used herein, the term “material” refers to an object such as a black object, namely an object which comprises carbon black and is composed of a polymer, e.g., black plastic. The object or material may or may not be an article of manufacture; it may also be shredded or cut polymeric material that is sorted in an amorphic or reduced form, as acceptable, for example, during certain sorting and recycling stages. Thus, according to the invention disclosed herein, unless otherwise stated or understood, the term “black plastic material” refers to a black plastic object, or to a black object in general.

The technique of the present invention enables automatic inspection and sorting of black plastic material(s) containing products progressing on a production line. A management system of the present invention, where the sorting data and the associated assigned certificate data are generated, based on the material inspection data, may be part of the inspection station or may be a stand-alone system in data communication with the inspection station. The sorting/certificate data can then be properly accessed and used at a sorting station downstream of the inspection station.

Life cycle of a plastic material refers to the period from manufacturing of the black material (as a virgin black plastic material or recycled black plastic material) until the next recycling of the black plastic material. Marking of the black plastic material may be already during its manufacturing or at any stage thereafter.

Production of black plastic products may utilize a composition comprising black carbon and a polymeric material or a prepolymer such as natural rubber or similar products and compositions of such natural products and one or more recycled plastic materials, wherein the natural plastic material is a plastic material which was not recycled (e.g., virgin) but used in a black product for the first time. In some cases, the recycled black plastic material may be set to include preselected concentrations of black plastic material which underwent recycling once, two or more times. To allow large scale recycling and reuse of specific plastic materials detection and identification of natural and recycled plastic materials is used.

Various plastic materials (e.g., polymeric materials) are marked during a recycling process (that is, during the production of recycled plastic material/product originating from used plastic products). Additionally, the black plastic material may be marked as a virgin plastic during its production or the production of black plastic products in which the virgin plastic is the main component.

The term “plastic” encompasses natural and non-natural or industrially manufactured polymers. Thus, the plastic materials may be polymers, such as Low-Density Polyethylene (LDPE), Linear Low-Density Polyethylene (LLDPE), High-Density Polyethylene (HDPE), Polypropylene (PP), Polyisoprenes, natural rubber (or latex) and other type of polymers.

In some embodiments, the article of manufacture of the invention or the object to be sorted comprises carbon black, rubber or a processed rubber and an amount of an XRF-identifiable material, as defined herein.

In some embodiments, the article of manufacture or the object to be sorted comprises recycled polymer (or plastic or rubber), unrecycled polymer (plastic or rubber), carbon black and an amount of an XRF-identifiable material, as defined herein.

The black plastic materials are marked by a specific marking (marker elements) that are embedded in the plastic materials. The markers may emit an electromagnetic signal which may be detected by a suitable spectrometer (reader). In an example, the markers emit a signal in response to incoming electromagnetic radiation, for example, UV, X-ray diffraction (XRD), or X-ray fluorescence (XRF) markers. In the description below, the use of XRF technique is exemplified regarding readings of the black plastic material signature in order to determine the black material properties/conditions and with regard to marking the black plastic material in accordance with its sorting data and certificate. It should however be understood that the principles of the novel approach of the present invention are not limited to this specific type of signature/marking.

XRF markers may be detected and measured by X-Ray Fluorescence (XRF) analysis by XRF spectrometers (readers) which may detect and identify their response (signature) signals. In an example, the XRF readers are Energy Dispersive X-Ray fluorescence EDXRF spectrometers. XRF markers are flexible, namely, they may be combined, blended or form compounds with, or embedded within a huge range of carriers, materials, substances, and substrates, without negatively affecting their signature signals.

The XRF markers may be, for example, in the form of inorganic salts, metal oxides, bi or tri metal atom molecules, polyatomic ions, and organometallic molecules (as described for instance in PCT/IL2020/050794 and PCT/IL2020/050793 which are incorporated herein by reference). In an example, XRF markers may be blended or applied to inorganic material (e.g., metals) or with organic (e.g. polymeric) materials, as described in WO 2018/069917 which is incorporated herein by reference. Due to this flexibility XRF markers, or a marking composition including several XRF markers (possibly with additional materials, such as carriers or additives), may be designed to have a preselected set of properties. Additionally, XRF marking can be detected and identified also when markers are present under the surface of an object but not on the surface itself, for instance, when the object is covered by a packaging material, dirt, or dust. Furthermore, XRF analysis enables measurement of the concentration of the markers present within a material as well as the ratio (the relative concentration) of the markers within a material.

The present invention provides a novel approach for overcoming problems relating to recycling and reuse of black plastic materials. In particular, the present invention enables the marking and identification of virgin black polymeric or black material polymers, such as natural polymers as rubber, and recycled plastic materials. Moreover, the technique of the present invention allows one to identify the number of times the polymeric material has undergone recycling. Furthermore, in case of a black product which includes both black virgin material(s) and black recycled plastic material, one is able to determine the composition of the product, namely, to measure a relation (e.g., ratio) between the virgin material, plastic material recycled once, plastic material recycled twice, and so on. To this end a set of one or more markers are introduced to the recycled material in each round of a recycling process during the overall recycling processes. Additionally, according to the invention, a virgin material may also be marked by one or more markers which may be introduced into the virgin material, for example, during its manufacturing or during the polymerization process, the compounding process, or during hot melt processing (e.g., extrusion) for instance during a production of a product containing the virgin material.

The one or more markers are embedded within a plastic material to obtain a marked black plastic material and may be detected and identified (e.g., by XRF analysis) at any stage during the life cycle of the marked plastic material, e.g., in the physical form of pellets, or as a component of a product, and during and after production of the product.

Thus, according to another broad aspect of the invention, it provides a method for providing an XRF-identifiable black polymeric raw material, such as natural rubber, the method comprising marking a sample of the polymeric raw material with an amount of an XRF-identifiable marker and black carbon, the amount of the XRF-identifiable marker defining an electromagnetic radiation signature indicative of the raw material composition and/or production profile (the raw material data). The profile may include one or more date of manufacture, site of manufacture, composition, presence or absence of unnatural additives, etc.

As known in the art, natural rubber is made by extracting a liquid sap, latex, from certain types of trees, mainly from Hevea brasiliensis trees, or the aptly named rubber tree. Latex is gathered from the trees by making a cut in the bark and collecting the runny sap in cups. This process is called tapping. To prevent the sap from solidifying, ammonia may be added. Acid is then added to the mix to extract the rubber, in a process called coagulation. The mixture is then passed through rollers to remove excess water, and ay thereafter be shredded, cut and washed to remove impurities. Once this is complete, the layers of rubber are hung over racks in smokehouses or left to air dry. Several days later, they will then be folded into bales ready for processing.

In accordance with the present invention, the rubber may be marked as detailed herein with an XRF-identifiable marker and the carbon black material at any stage of its production. Where the rubber is mixed with at least one another material, the rubber is marked prior to mixing with the at least one another material.

Marking may be during the stage of latex collection, i.e., during tapping; prior to, during or after sap solidification with a solidification agent; prior to, during or after coagulation; or after the rubber is dried.

The invention also provides a method of sorting black materials in a recycling process, the method comprising:
providing measured data indicative of an electromagnetic radiation signature embedded in a black material;
identifying radiation emitted (secondary radiation) from said material in response to X-Ray or gamma-ray (primary radiation), said radiation having spectral features (i.e., peaks in a particular energy/wavelength) characteristic of the signature, thereby identifying presence of the black material.

The invention further provides a method of managing black material recycling process, the method comprising:
providing first measured data indicative of one or more first electromagnetic radiation signatures embedded in one or more black plastic materials in a product;
analyzing the measured data to determine, for each of said one or more black plastic materials, a respective plastic material condition data, wherein the respective plastic material condition data is indicative of preceding use of said plastic material;
generating first sorting data for each of said one or more black plastic materials, based on the respective plastic material condition; and
generating marking data for at least one of said one or more black plastic materials, based on the first sorting data, wherein the marking data includes data indicative of at least one marker to be introduced into each of said one or more plastic materials to provide electromagnetic radiation signal for managing a recycling process said one or more black plastic material.

In some embodiments, the method further comprises utilizing at least one of the black plastic material condition data and the sorting data of said plastic material and generating and storing certificate data characterizing a current condition of said black plastic material to be sorted.

The data indicative of the at least one marker may be obtained from a database, storing, for each plastic material reuse type, data indicative of a life cycle of said plastic material in association with matching data about corresponding one or more markers.

The data indicative of the at least one marker may comprise data corresponding to (a) a number of a successive life cycle of said plastic material being recycled and (b) a successive product type for reuse of recycled plastic material.

In some embodiments, the black plastic material condition data is indicative of a relation between said black plastic material and a predetermined black virgin material contained in the product. For example, the first measured data also comprises data indicative of one or more electromagnetic radiation signatures of said predetermined natural material, as defined herein.

The at least one marker may be introduced into the plastic material in a single package together with the carbon black and other additional additives in a single masterbatch, as disclosed herein.

In some embodiments, the method further comprises providing second measured data indicative of one or more second electromagnetic radiation signals originated by one or more contaminant elements presented in the plastic material after being sorted by introducing said marking therein.

In some embodiments, the method further comprises providing second measured data indicative of one or more second electromagnetic radiation signals originated by one or more contaminant elements presented in the black plastic material after being sorted by introducing said marking therein and updating the certificate data characterizing the black plastic material.

The electromagnetic radiation signals of the measured data may be of at least one of the following types: UV signals; X-Ray Diffraction (XRD) signals; X-Ray Fluorescence (XRF) signals.

In some embodiments, the electromagnetic radiation signals of the measured data comprise X-Ray Fluorescence (XRF) signals; and the data indicative of the at least one marker correspond to the at least one marker responding by XRF response signals to XRF exciting radiation.

According to another broad aspect of the invention, it provides a method for managing a black material recycling process comprising:
providing black plastic material condition data indicative, for each of one or more plastic materials in a product, of preceding use of said plastic material in association with one or more plastic product types;
analyzing the plastic material condition data and generating sorting data for each of said one or more plastic materials, based on the respective plastic material condition;
generating marking data for at least one of said one or more plastic materials, based on the sorting data, wherein the marking data includes at least one XRF marker to be introduced into each of said one or more black plastic materials to provide electromagnetic radiation signal for managing a recycling process of the black plastic material; and
utilizing at least one of the black plastic material condition data and the sorting data of said plastic material and generating and storing certificate data charactering a current condition of said black plastic material to be sorted.

Also provided is a method for identifying a black plastic during sorting of plastic materials, the method comprising:
irradiating with X-Ray or Gamma-Ray radiation a collection of plastic objects comprising black objects marked with at least one XRF-identifiable marker;
detecting an X-Ray or Gamma-Ray signal arriving from the objects in response to the X-Ray or Gamma-Ray radiation applied thereto;
applying spectral processing to the detected radiation signal to obtain data indicative of the presence, absence or any change in the predefined characteristic relating to the black plastic.

In some embodiments, the method comprises:
simultaneously irradiating a plurality of objects with at least one X-ray or Gamma-ray excitation beam having a spatially distributed modulated intensity; wherein the intensity of the beam arriving at each of the objects is different and identifiable and wherein the plurality of objects comprising black objects;
detecting a secondary X-ray radiation arriving from the plurality of objects and generating signals indicative of the spatial intensity distribution on the plurality of objects; and
identifying which of the plurality of black objects are marked by a marking composition according to the detected spatial intensity distribution.

The invention further provides a method comprising:
simultaneously irradiating a plurality of objects with at least one X-ray or Gamma-ray excitation beam having a spatially distributed modulated intensity; wherein the intensity of the beam arriving at each of the objects is different and identifiable and wherein the plurality of objects comprising black objects;
detecting a secondary X-ray radiation arriving from the plurality of objects and generating signals indicative of the spatial intensity distribution on the plurality of objects; and
identifying which of the plurality of black objects are marked by a marking composition according to the detected spatial intensity distribution.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are graphs showing intensity as function of concentration for the different components in marker system A in the carbon black powder before pelletizing.

FIGS. 2A-2C are graphs showing intensity as function of concentration for the different components in marker system B in the carbon black powder before pelletizing.

FIGS. 3A-3C are graphs showing intensity as function of concentration for the 3 combinations of marker system A after pelletizing.

FIGS. 4A-4C Peak intensity as function of concentration for pelletized CB vs. powder CB: Marker system A

FIGS. 5A-5C are graphs showing B-Parts intensity as function of pelletized CB—Marker system.

FIGS. 6A-6C are graphs showing peak intensity as function of concentration for pelletized CB vs. powder CB: Marker system B.

FIGS. 7A-7C are graphs showing intensity as function of concentration in CB MB for the different components in marker system A.

FIGS. 8A-8C are graphs showing intensity as function of concentration in CB MB for the different components in marker system B.

FIG. 9 is a graph showing red spectrum—peak signal for the three components of marker system B in thick sample containing 0.5 wt % CB MB loading. Black—unmarked sample.

FIG. 10 is a blue spectrum—peak signal for the three components of marker system B in single foil layer containing 2 wt % CB MB loading. Black—unmarked sample.

### DETAILED DESCRIPTION OF EMBODIMENTS

The present disclosure relates to means and methods for marking/identifying black polymers products and is based on the development of specific markers/identifiable components that utilize X-ray fluorescence (herein: “XRF”), which enables identification and sorting of black plastics for recycling purposes.

The specific markings/identifiable components denoted herein XRF detectable/identifiable markers are added (incorporated) during the process of black plastic manufacture.

As shown in the examples below, the XRF-detectable/identifiable markers remained both stable and active (i.e. detectable) during the entire black plastic manufacturing process. Accordingly, XRF-detectable identifiable markers can be added in each one of the black plastic manufacturing steps, including, inter alia, in a dry blending step, in a pelletizing step, in compounding (i.e. masterbatch production) step, in a blowing step or in an injection molding step. This results in a wide range of XRF-identifiable intermediate products (e.g. powder, pelletized powder or masterbatch) as well as plastic products.

In accordance with the first of its aspects, the present disclosure provides a XRF-identifiable carbon black powder comprising carbon black and at least one XRF identifiable marker.

Powder as used herein in reference to the XRF-identifiable carbon black relates to fine, dry particles having a size of at most about 100 nm. Additionally, the particles may refer to a dry blend of at least one carbon black and at least one XRF identifiable marker.

In accordance with some embodiments, the XRF-identifiable carbon black powder is for use in the preparation of XRF-identifiable carbon black pelletized powder. In accordance with some further embodiments, the XRF-identifiable carbon black powder is subjected to a pelletizing process. In some embodiments, pelletizing the dry blend is by a wet pelletizing process to obtain the XRF-identifiable carbon black pelletized powder.

As appreciated by those versed in the field, the XRF-identifiable carbon black powder is subjected to pelletizing, for example, in order to coagulate the powder.

In accordance with some other aspects, the present disclosure provides an XRF-identifiable carbon black pelletized powder comprising a homogenous blend of carbon black and at least one XRF identifiable marker.

The XRF-identifiable marker in accordance with the present invention is a substance which includes at least one compound or element identifiable by XRF signature, namely, can be identified by XRF analysis (e.g., by an XRF analyzer), XRF analysis, that is analysis of the response X-ray signal, can be carried out by a suitable spectrometer such as XRF analyzer which may operate in uncontrolled environment without vacuum conditions (e.g. energy dispersive XRF analyzer which may be a benchtop, mobile or handheld device).

In some embodiments, the XRF-identifiable marker is a material having a XRF signature and may be selected in a form which includes one or more elements that are identifiable by XRF.

In some embodiments, the XRF-identifiable marker is or comprises at least one element of the periodic table of the elements which in response to x-ray or gamma-ray (primary radiation) radiation emits an x-ray signal (secondary radiation) with spectral features (i.e. peaks in a particular energy/wavelength) characteristic of the element (an x-ray response signal as XRF signature). An element having such response signal is considered XRF-sensitive.

The XRF signature may depend on the marking(s) (material compositions, concentrations, etc.) as well as the surface/structure of the specific product on or in which the markings has been embedded.

The XRF-identifiable marker may be in the form of salts or may be a material comprising at least one atom.

In some embodiments, the XRF-identifiable marker is or comprises at least one atom or comprises at least one atom selected from, Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La and Ce.

In some embodiments, the XRF-identifiable marker is or comprises at least one metal atom.

In some other embodiments, the XRF-identifiable marker comprises at least one metal salt or a material comprising at least one metal atom.

In some embodiments, the XRF-identifiable marker is an atom or comprises at least one atom selected from Mo, Ag, Cr, Ti, Mn, K, Ca, Sc, V, Co, Ni, Zn, Ge, Rb, Sr, Y, Zr, Nb, Mo, Cd and In.

In some embodiments, the XRF-identifiable marker is a material comprising at least one atom selected from Mo, Ag, Cr, Ti, Mn, K, Ca, Sc, V, Co, Ni, Zn, Ge, Rb, Sr, Y, Zr, Nb, Mo, Cd and In.

In some embodiments, the XRF-identifiable marker is at least one atom or comprises at least one atom selected from Mo, Ag, Cr, Ti and Mn.

In some embodiments, the XRF-identifiable marker is a material comprising at least one atom selected from Mo, Ag, Cr, Ti and Mn.

In some embodiments, the XRF-identifiable marker is at least one metal atom within a carrier. In some embodiments, the XRF-identifiable marker is at least one metal atom within nanoparticles. In some embodiments, the XRF-identifiable marker is or comprises an Ag atom within nanoparticles.

In some other embodiments, the XRF-identifiable marker is or comprise at least one non-metal atom. In some other embodiments, the XRF-identifiable marker is or comprise at least one atom of P, Se, Br, S, Cl, I and Si.

In some embodiments, the XRF-identifiable marker is in the form of at least one of molybdenum disulfide, zinc oxide, manganese stearate, manganic oxide, manganese chloride, zinc diricinoleate, potassium bromide, chromium oxide, sodium bromide, titanium oxide, titanium nitride, ammonium bromide and calcium butyrate.

In some embodiments, the XRF-identifiable marker is in the form of at least one of zinc oxide, manganese stearate, manganese chloride, potassium bromide, chromium oxide, molybdenum disulfide, sodium bromide, titanium oxide, manganic oxide, titanium nitride, ammonium bromide and calcium butyrate.

In some embodiments, the XRF-identifiable marker is in the form of at least one, at least two or three of titanium oxide, molybdenum disulfide and silver atom.

In some embodiments, the XRF-identifiable marker is in the form of at least one, at least two or three of titanium oxide, manganic oxide and chromium oxide.

As described herein, the XRF-identifiable marker is mixed with a carbon black.

The amounts of the carbon black and the at least one XRF-identifiable marker in the identifiable carbon black may vary depending for example, on the end plastic product. Unless otherwise indicated, the amount of at least one XRF-identifiable marker in the identifiable carbon black or any ration thereof refers to the amount or ratio thereof of the active element in the XRF-identifiable marker. In other words, in cases where the XRF-identifiable marker is provided as a salt, for example, a metal salt, the amount of the XRF-identifiable marker or any ratio thereof is made in reference to the active element, i.e. the metal atom.

Generally, the lower the ratio between the carbon black and the at least one XRF-identifiable marker, the higher the XRF-identifiable marker loading and hence the detection is improved.

In some embodiments, the ratio between carbon black and the at least one XRF-identifiable marker in the pelletized product or in a composition of the invention is at least 100:1, respectively, or 200:1, 300:1, 400:1, 500:1, 600:1, 700:1, 800:1 or 900:1.

In some other embodiments, the ratio between carbon black and the at least one XRF marker in the pelletized product is between about 100:1 and about 1000:1, respectively.

The XRF-identifiable carbon black pelletized powder comprising a homogenous blend of the carbon black and of the at least one XRF identifiable marker can be of any size or shape. For example, the pelletized powder is in a form of pellets with sizes ranging between about 30 and about 200 grains.

As described herein, the XRF-identifiable carbon black pelletized powder may be in accordance with some embodiments, produced by a pelletizing process.

In accordance with the present disclosure, the XRF-identifiable carbon black, being for example in the form of pelletized powder, is for use in a compounding process to obtain a masterbatch mixture. In some embodiments, the XRF-identifiable carbon black pelletized powder for use in preparing a masterbatch mixture

In accordance with some other aspects, the present disclosure provides an XRF-identifiable masterbatch (MB) mixture comprising a homogenous blend including carbon black, at least one XRF identifiable marker and at least one thermoplastic polymer.

The XRF-identifiable masterbatch (MB) mixture may be produced by using a XRF-identifiable carbon black or alternatively by compounding carbon black, at least one XRF identifiable marker and at least one thermoplastic polymer. In other words, the masterbatch mixture in accordance with the present disclosure may be obtained by either a XRF-identifiable carbon black compounded with at least one thermoplastic polymer formed a-priori or alternatively by compounding the three components individually.

The amounts of the at least one XRF-identifiable marker in the XRF-identifiable masterbatch mixture may vary. In some embodiments, the marked masterbatch comprises at least 0.05% w/w of the at least one XRF-identifiable marker, at times at least 0.08% w/w, at times at least 0.1% w/w, at times at least 2% w/w, at times at least 3% and at times at least 5% of the at least one XRF-identifiable marker.

In some embodiments, the marked masterbatch comprises between about 0.05% w/w to about 5% of the at least one XRF-identifiable marker, at times between about 0.1% w/w and about 4% w/w, at times between about 0.5% w/w and about 3% and at times between about 0.5% w/w and about 2% of the at least one XRF-identifiable marker.

In some embodiments, the XRF-identifiable masterbatch mixture comprising at least about 20%, at times at least about 30%, at times at least about 40% and at times at least about 50% of a thermoplastic polymer. In some embodiments, the XRF-identifiable masterbatch mixture comprising about 40% of a thermoplastic polymer.

As used herein, the term “polymer” should be understood as having the general meaning known by those skilled in art. Although not limited to, the polymer utilized according to the invention may be a plastic material. In some embodiments, the polymer is a thermoplastic polymer, i.e., exhibits a property in which a solid or essentially solid material turns upon heating into a hot flowable material and reversibly solidifies when sufficiently cooled. The term also denotes that the material has a temperature or a temperature range at which it becomes a hot flowable material.

In some embodiments, the polymer is selected from polyolefins, polyamides, polystyrenes, polyesters, polycarbonates, polyethylene terephthalates, polyurethanes, polyamides, polyimides, polyacrylonitriles polyvinyl alcohols and biaxially oriented polymer.

In some embodiments, the polymer is selected from polyolefins (e.g. high density polyethylene (HDPE), low density polyethylene (LDPE), polypropylene (PP)); polyethylene terephthalate (PET); polystyrene (PS); polyvinylchloride (PVC); polyurethane (PU); polyamides (PA); polyacrylonitriles; polyimides; polyvinyl alcohols and biaxially oriented polymer.

In such embodiments, the polyolefin is selected from polypropylene and polyethylene.

In some embodiments, the polymer is a polyethylene. In some other embodiments, the polymer is low density polyethylene (LDPE).

The masterbatch of the present disclosure may be in the form of liquid, particle matter, particles or the like provided that it comprises a homogenous blend of the components. Hence, in accordance with the present disclosure, the XRF-identifiable marker may be incorporated into the at least one polymer (polymeric element) without substantially affecting the physical properties (i.e., optical and mechanical properties) of same polymer free of XRF-identifiable marker.

When referring to the XRF-identifiable marker being incorporated into the at least one polymer it is to be understood that the polymer and the at least one XRF-identifiable marker are being intimately held together by physical interactions therebetween. It was suggested that this allows the at least one XRF-identifiable marker to be homogenously distributed within the polymer, thereby contributing to the increased XRF signal.

The masterbatch mixture can include additional components, such as non-polymeric components. In some embodiments, the masterbatch mixture comprises an antioxidant, a UV-stabilizer, a flame retardant, a pigment, a stabilizer and a wetting agent.

In some embodiments, the masterbatch is in the form of particulate matter comprises particles. In some embodiments, the masterbatch is in the form of pellets. In some embodiments, each particle comprises a blend of at least one XRF-identifiable marker, a carbon black and at least one thermoplastic polymer.

In accordance with the present disclosure, XRF-identifiable masterbatch mixture can be used for the preparation of an article of manufacture by using for example any manufacture method known in the art. In some embodiments, the XRF-identifiable masterbatch mixture is for use in preparing an article of manufacture.

Thus, in some other aspects the present disclosure provides an XRF-identifiable article of manufacture comprising a homogenous blend comprising carbon black, at least one XRF identifiable marker and at least one thermoplastic polymer.

The article of manufacture in accordance with the present disclosure may be any plastic product, for example but not limited to plastic products used in the food industry (e.g. packing or equipment), in agriculture (e.g. tools, buckets or films), cosmetic industry (e.g. bottles) or automobile industry (e.g. tiers).

The article of manufacture comprises may comprise varying amounts of the at least one XRF identifiable marker, depending, for example on the size, shape of the article. In some embodiments, the article of manufacture comprises at least 2 ppm, at times at least at least 4 ppm, at times at least 8 ppm, at times at least 12 ppm, at times at least 16 ppm, at times at least 20 ppm, at times at least 24 ppm, at times at least 41 ppm, at times at least 50 ppm, at times at least 60 ppm and at times at least 500 ppm of the at least one XRF identifiable marker.

In some embodiments, the article of manufacture comprises between about 2 ppm and about 500 ppm of the at least one XRF identifiable marker, at times between about 4 ppm and about 60 ppm, at times between about 4 ppm and about 50 ppm, at times between about 8 ppm and about 41 ppm of the at least one XRF identifiable marker.

As further shown in the examples below, it was possible to differentiate marked black plastic from unmarked black plastic. Specifically, the results show the marking of the present invention using the at least one XRF identifiable marker is effective in a variety of articles of manufacture, including thick samples and thin samples.

As appreciate, the article of manufacture may be obtained by any method known in the art, including, for example, injection molding or blowing. As also appreciated, the process for the preparation of the article of manufacture comprises “diluting” a masterbatch mixture, for example, the XRF-identifiable masterbatch mixture of the present disclosure with at least one thermoplastic polymer. The at least one thermoplastic polymer that is added during preparation of the article of manufacture may be the same polymer as in the masterbatch mixture or may be a different polymer. In accordance with some embodiments, the polymer is the masterbatch mixture and the polymer added during preparation of the article of manufacture are at least compatible, at times identical.

The present disclosure provides in accordance with some aspects, a method of preparing an XRF identifiable article of manufacture, the method comprising:
(i) pelletizing a mixture comprising carbon black and at least one XRF identifiable marker;
(ii) melt blending pellets obtained from said pelletizing, with at least one thermoplastic polymer to form a molten;
(iii) molding the molten to obtain said article of manufacture.

### NON-LIMITING EXAMPLES

### Materials and Methods

Samples of bare Carbon Black (CB) Printex 60A powder and black products were initially received for background characterization. Based on the analysis results, two markers system were designed denoted herein as “A” and “B”. Each marker system comprised a sequence of three components and tested at three different concentrations, total of 6 samples.

Marker A comprises MoS₂, Silver NP and TiN and Marker B comprises TiN, Cr₂O₃ and Mn₂O₃.

Three combinations of each one of marker A and marker B were tested, such that three different combinations at different amounts of the three components in each combination were mixed with CB.

The following Tables 1 and 2 show details of the marker A and marker B.

TABLE 1
amounts of the components of marker A and CB

| | Marker (g) | CB (g) |
|---|---|---|
| 1^{st} combination | | |
| MoS₂ | 6.673 | 1984.156 |
| Silver NP | 4.000 | 1984.156 |
| TiN | 5.170 | 1984.156 |
| 2^{nd} combination | | |
| MoS₂ | 10.10 | 1976.235 |
| Silver NP | 6 | 1976.235 |
| TiN | 7.756 | 1976.235 |
| 3^{rd} combination | | |
| MoS₂ | 16.683 | 1960.391 |
| Silver NP | 10.00 | 1960.391 |
| TiN | 12.926 | 1960.391 |

TABLE 2
amounts of the components of marker B and CB

| | Marker (g) | CB (g) |
|---|---|---|
| 1^{st} combination | | |
| TiN | 5.170 | 1986.235 |
| Cr₂O₃ | 5.846 | 1986.235 |
| Mn₂O₃ | 5.747 | 1986.235 |
| 2^{nd} combination | | |
| TiN | 7.756 | 1974.85 |
| Cr₂O₃ | 8.769 | 1974.85 |
| Mn₂O₃ | 8.621 | 1974.85 |
| 3^{rd} combination | | |
| TiN | 12.926 | 1958 |
| Cr₂O₃ | 14.616 | 1958 |
| Mn₂O₃ | 14.368 | 1958 |

When referring to the active element in the marker, as can be seen in Table 3, the first combination in both marker A and marker B included 2000 ppm of each component, the second combination in both marker A and marker B included 3000 ppm of each component and the third combination in both marker A and marker B included 5000 ppm of each component.

TABLE 3
Various combinations of tested active
element in marker A and marker B

| | Marker A | | | |
|---|---|---|---|---|
| | MoS₂ | Silver NP*, ** | TiN | CB |
| | (ppm)* | (ppm) | (ppm)* | (ppm) |
| Combination # 1 | 2000 | 2000 | 2000 | 992078 |
| Combination # 2 | 3000 | 3000 | 3000 | 988117 |
| Combination # 3 | 5000 | 5000 | 5000 | 980196 |
| Reference | | | | 2000 |

*the active element is Mo, Ag and Ti and the amount provided in ppm
correspond to the amount of the active element in the component,
**NP-nanoparticles

Marker B

| | TiN* | Cr₂O₃* | Mn₂O₃* | CB |
|---|---|---|---|---|
| | (ppm) | (ppm) | (ppm) | (ppm) |
| Combination # 4 | 2000 | 2000 | 2000 | 991618 |
| Combination # 5 | 3000 | 3000 | 3000 | 987427 |
| Combination # 6 | 5000 | 5000 | 5000 | 979045 |

*the active element is Ti, Cr and Mn and the amount provided in ppm correspond to the amount of the active element in the component.

After finalizing the different loadings (conc.1, conc.2, conc.3 for each marker system), the six markers combinations were mechanically mixed for approx. 5 minutes with CB powder (batch size: 2 kg each) at the amounts detailed in Table 1 and were subjected to a standard pelletizing step. The marked pelletized CB samples were then compounded with low-density polyethylene (LDPE) and loading instructions were sent for each combination to compensate on markers' addition. Table 4 shows the theoretical loading to compensate on markers' addition (originally 40 wt % CB is added) and actual loading which was added experimentally. As can be seen, the actual marked CB loading in all the samples was 40% regardless of the marker system concentration indicating that the markers' loading in the MB is lower than anticipated.

TABLE 4
CB loading in MB production

| | Theoretical | | Experimental | |
|---|---|---|---|---|
| | Loading (%) | | Actual Loading (%) | |
| | Marked | LDPE | Marked | LDPE |
| Sample Description | CB (wt %) | (wt) | CB (%) | (%) |
| Marker system A - Conc. 1 | 40.3195 | 59.685 | 40.000 | 60.00 |
| Marker system A - Conc. 2 | 40.4811 | 59.519 | 40.000 | 60.00 |
| Marker system A - Conc. 3 | 40.8083 | 59.197 | 40.000 | 60.00 |
| Marker system B - Conc. 1 | 40.3382 | 59.668 | 40.000 | 60.00 |
| Marker system B - Conc. 2 | 40.5093 | 59.497 | 40.000 | 60.00 |
| Marker system B - Conc. 3 | 40.8560 | 59.140 | 40.000 | 60.00 |
| Reference | | 60.000 | 40.000 | 60.00 |

When referring to the active element in the marker, the first combination in both marker A and marker B included 806 ppm of each component, the second combination in both marker A and marker B included 1210 ppm of each component and the third combination in both marker A and marker B included 2016 ppm of each component.

Next, all the above 7 CB MBs were mixed with LDPE resin at 0.5, 1, and 2 wt % and processed to produce 21 injection molded samples+21 foil samples for SMX detection, total 42 samples were produced. The compositions of the samples are shown in the Table below.

TABLE 5
Final products' composition

| | MB loading in the final | | |
|---|---|---|---|
| | product (wt %) | | |
| Marked CB-MB A - Conc. 1 | 0.5 | 1 | 2 |
| Marked CB-MB A - Conc. 2 | 0.5 | 1 | 2 |
| Marked CB-MB A - Conc. 3 | 0.5 | 1 | 2 |
| Marked CB-MB B - Conc. 1 | 0.5 | 1 | 2 |
| Marked CB-MB B - Conc. 2 | 0.5 | 1 | 2 |
| Marked CB-MB B - Conc. 3 | 0.5 | 1 | 2 |
| Reference (unmarked CB-MB) | 0.5 | 1 | 2 |

TABLE 6
Final products' composition

| | active element in final | | |
|---|---|---|---|
| | product (ppm) | | |
| marker A - Conc. 1 for each component | 4 | 8 | 16 |
| marker A - Conc. 2 for each component | 6 | 12 | 24 |
| marker A - Conc. 3 for each component | 10 | 20 | 41 |
| marker B - Conc. 1 for each component | 4 | 8 | 16 |
| marker B - Conc. 2 for each component | 6 | 12 | 24 |
| marker B - Conc. 3 for each component | 10 | 20 | 41 |
| Reference | 0 | 0 | 0 |

### Results

### Dry Mixing Step

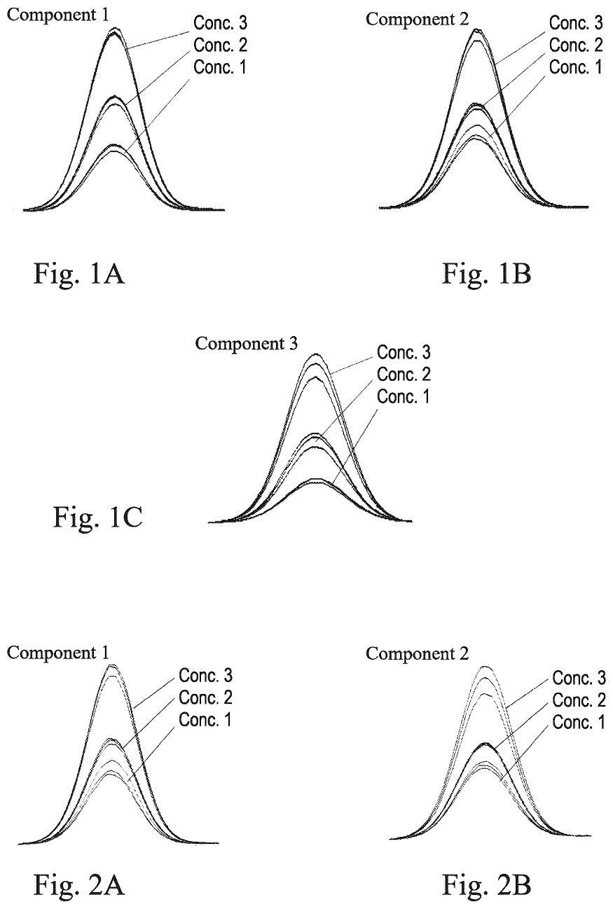

Bare components at their powder form were mechanically mixed with CB powder for approximately 5 minutes. Each concentration was measured 3 times for homogeneity evaluation. The detection results for the three concentrations of marker system A are shown in Table 7 and FIG. 1.

TABLE 7
Detection results for the 3 combinations of marker system A

| | Marker System A | | | | | | | | |
|---|---|---|---|---|---|---|---|---|---|
| | Component 1 | | | Component 2 | | | Component 3 | | |
| | Average | STD | R. STD | Average | STD | R. STD | Average | STD | R. STD |
| | (a.u) | (a.u) | (%) | (a.u) | (a.u) | (%) | (a.u) | (a.u) | (%) |
| Conc. 1 | 383047.3 | 21886.21 | 6 | 141771 | 19356.16 | 14 | 476028.7 | 34292.45 | 7 |
| Conc. 2 | 657588.3 | 25599.25 | 4 | 219447 | 15451.3 | 7 | 894815.7 | 13221.44 | 1 |
| Conc. 3 | 1039791 | 16149.19 | 2 | 353790 | 17420.78 | 5 | 1288623 | 8265.02 | 1 |

Considering that the bare marker components were mixed with the CB powder for only few minutes, all the three components showed distinguish peaks and all concentrations can be separated from each other. The relative STD (=100*std/average), which is indication for homogeneity, is considerably low for all the three components suggesting good homogeneity of markers' component in the CB powder.

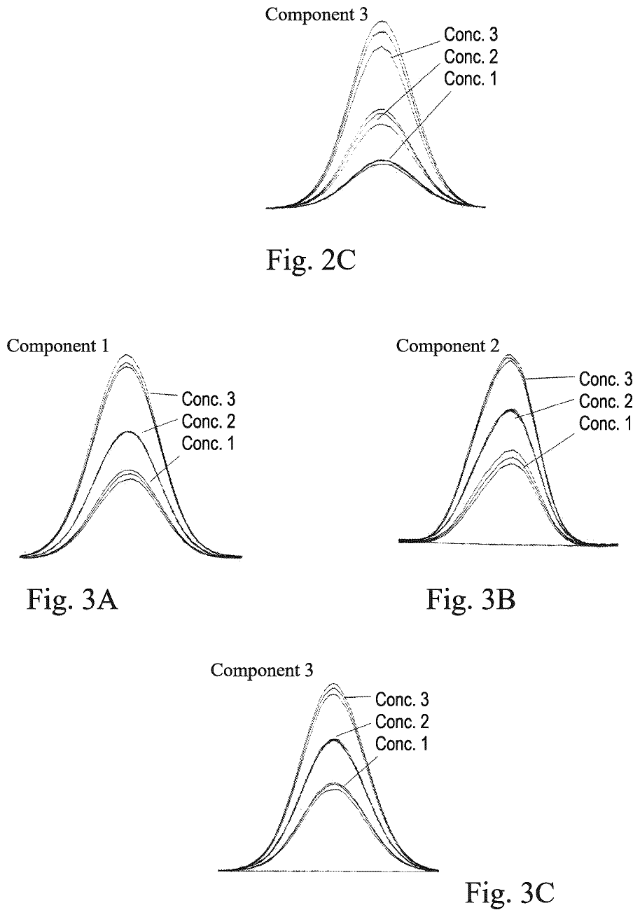

The detection results for the different components for marker system B are shown in Table 8 and FIG. 2. Same here, each concentration was measured 3 times for homogeneity evaluation.

TABLE 8
Detection results for the 3 combinations of marker system B

| | Marker System B | | | | | | | | |
|---|---|---|---|---|---|---|---|---|---|
| | Component 1 | | | Component 2 | | | Component 3 | | |
| | Average | STD | r. STD | Average | STD | r. STD | Average | STD | r. STD |
| | (a.u) | (a.u) | (%) | (a.u) | (a.u) | (%) | (a.u) | (a.u) | (%) |
| concentration 1 | 592443.7 | 54622.35 | 9 | 778458 | 39045.93 | 5 | 517802 | 20099.81 | 4 |
| concentration 2 | 819181.7 | 19914.93 | 2 | 1012437 | 5011.14 | 0.5 | 982201.3 | 72448.68 | 7 |
| concentration 3 | 1398773 | 64262.25 | 5 | 1701970 | 164035.8 | 10 | 1829376 | 154316.1 | 8 |

All the three components in marker system B showed clear peaks. Same as shown in marker system A, also marker system B presented distinguish peaks in each concentration and all peaks were well separated from each other. However, when comparing the two marker systems, marker system B showed lower relative STD values in all the concentrations, suggesting that marker system B has potentially better distribution in CB powder.

### Pelletizing Step

All components were analyzed after pelletizing to evaluate the quality of dispersion. From each concentration 3 measurements were taken and results for marker system A are shown in Table 9 and FIG. 3. As evident from the results, all components showed relative STD below 10 in all the concentrations, indication of good dispersion quality.

TABLE 9
Detection results for the 3 combinations of marker system A after pelletizing step

| | Marker System A | | | | | | | | |
|---|---|---|---|---|---|---|---|---|---|
| | Component 1 | | | Component 2 | | | Component 3 | | |
| | Average | STD | R. STD | Average | STD | R. STD | Average | STD | R. STD |
| | (a.u) | (a.u) | (%) | (a.u) | (a.u) | (%) | (a.u) | (a.u) | (%) |
| concentration 1 | 905317.7 | 42904 | 5 | 304808.3 | 26090 | 9 | 586227.3 | 14992.95 | 3 |
| concentration 2 | 1386094 | 2295 | 0.2 | 461442.3 | 5185.01 | 1 | 899036.7 | 28666.18 | 3 |
| concentration 3 | 2094822 | 52237 | 2 | 645441.7 | 13170.81 | 2 | 1265099 | 25730 | 2 |

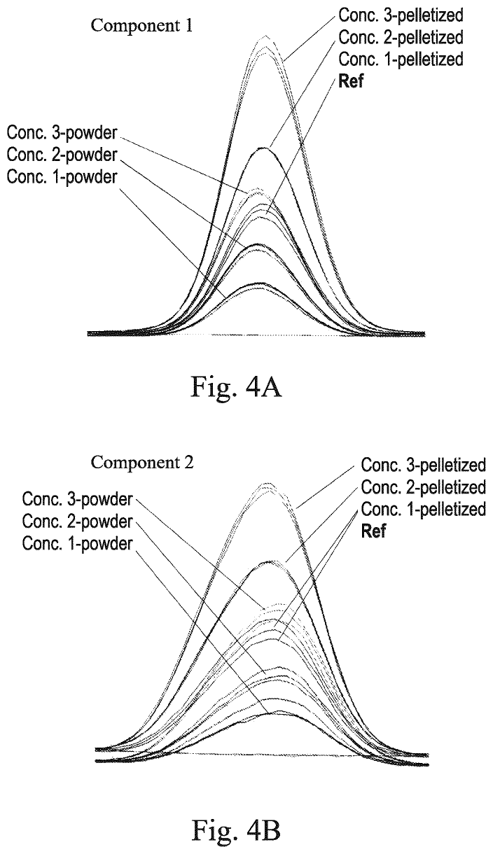

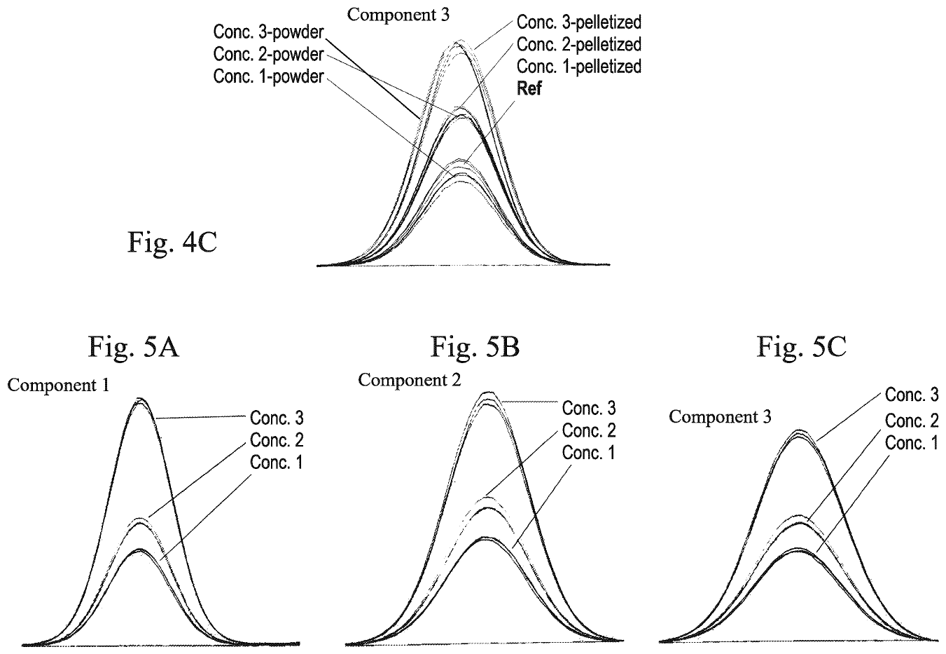

Evaluation of dispersion quality before and after pelletizing was also studied by comparing the components' intensity before pelletizing (powder form) and after pelletizing. The results are plotted in FIG. 4 where dark colors specify components after pelletizing and light colors specify components before pelletizing.

As shown in FIG. 4, both components 1 and 2 showed higher peak intensity after pelletizing suggesting an improvement in dispersion quality. Component 3 on the other hand did not present increase in peak intensity after pelletizing and it can be assumed that maximum dispersion already reached in the dry mixing step.

Same as done for marker system A, was repeated for marker system B and all components were analyzed after pelletizing to evaluate the quality of dispersion. From each concentration 3 measurements were taken and results for marker system B re shown Table 10 and FIG. 5. As shown in FIG. 5, all the three components in marker system B presented relative STD below 5 in all the concentrations, lower than the values obtained in marker system A. As the lower the relative standard deviation the better the dispersion quality in CB, it can be concluded that the dispersion quality of marker system B is superior than marker system A. This supports our previous claim that marker system B is more compatible with CB powder.

TABLE 10
Detection results for the 3 combinations of marker system B after pelletizing step

| | Marker System B | | | | | | | | |
|---|---|---|---|---|---|---|---|---|---|
| | Component 1 | | | Component 2 | | | Component 3 | | |
| | Average | STD | R. STD | Average | STD | R. STD | Average | STD | R. STD |
| | (a.u) | (a.u) | (%) | (a.u) | (a.u) | (%) | (a.u) | (a.u) | (%) |
| concentration 1 | 616915.3 | 13568.29 | 2 | 895194.7 | 27058.02 | 3 | 1076165 | 19190.31 | 2 |
| concentration 2 | 832225.3 | 23669.21 | 3 | 1256152 | 33297.48 | 3 | 1483002 | 35585.84 | 2 |
| concentration 3 | 1570542 | 17834.78 | 1 | 2179145 | 2181.62 | 0.1 | 2495610 | 5144.72 | 0.2 |

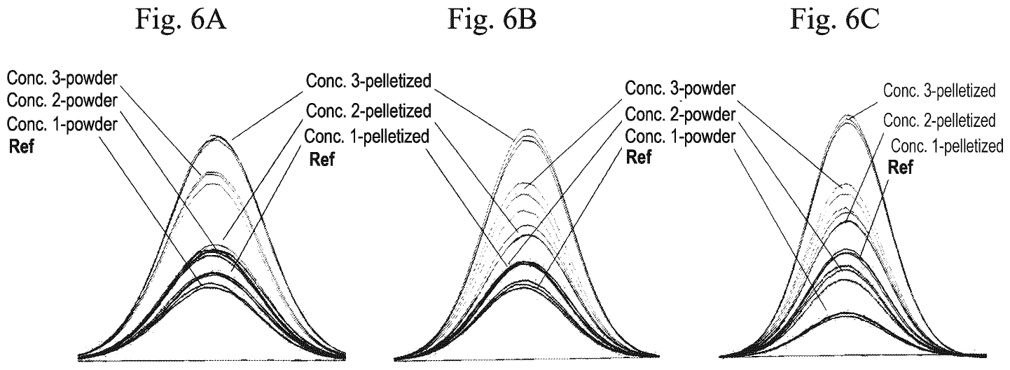

Evaluation of dispersion quality before and after pelletizing was also studied for marker system B and results are plotted in FIG. 6.

As can be seen, all the three components showed an increase in peak intensity after pelletizing suggesting that this step is essential to achieve high dispersion in CB.

Summarizing this step, pelletizing increases components detectability and decreases relative STD values, indication that the dispersion of all components in both systems was improved.

### Compounding Step

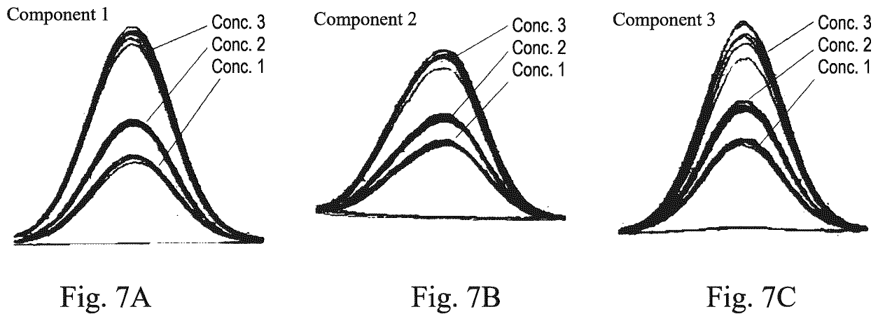

All pelletized CB were mixed at 40 wt % with 60 wt % LDPE and compounded to produce marked CB MB. The detection results for marked CB MB containing marker system A are shown in Table 11 and FIG. 7. As expected, with decreasing CB loading (from 100 to 40 wt % in MB), the average intensity for all the components decreases, however without major changes in the relative STD supporting again our observation that the resultant dispersion after pelletizing is good.

TABLE 11
Detection results for the 3 combinations of marker system A after compounding step

| | Marker System A | | | | | | | | |
|---|---|---|---|---|---|---|---|---|---|
| | Component 1 | | | Component 2 | | | Component 3 | | |
| | Average | STD | R. STD | Average | STD | R. STD | Average | STD | R. STD |
| | (a.u) | (a.u) | (%) | (a.u) | (a.u) | (%) | (a.u) | (a.u) | (%) |
| concentration 1 | 549082.3 | 11370.6 | 2 | 192561.2 | 3177.8 | 2 | 266865.7 | 4865.38 | 2 |
| concentration 2 | 772427.7 | 11158.84 | 1 | 254256.8 | 3547.7 | 1 | 371409 | 14965.36 | 4 |
| concentration 3 | 1357321 | 47939.58 | 4 | 420058.2 | 20987.2 | 5 | 582088.8 | 38002.7 | 7 |

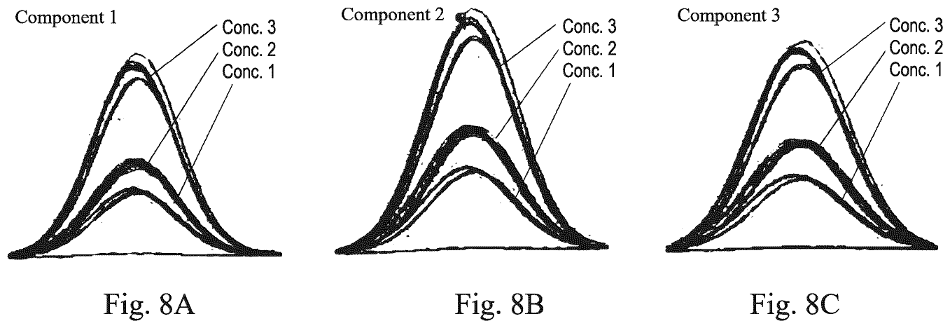

The detection results for marked CB MB containing marker system B are shown in Table 12 and FIG. 8. Same as observed with marker system A, with decreasing CB loading (from 100 to 40 wt % in MB), the average intensity for all the components of marker system B decreases without major changes in the relative STD.

TABLE 12
Detection results for the 3 combinations of marker system B after compounding step

| | Marker System B | | | | | | | | |
|---|---|---|---|---|---|---|---|---|---|
| | Component 1 | | | Component 2 | | | Component 3 | | |
| | Average | STD | R. STD | Average | STD | R. STD | Average | STD | R. STD |
| | (a.u) | (a.u) | (%) | (a.u) | (a.u) | (%) | (a.u) | (a.u) | (%) |
| concentration 1 | 259056.5 | 8827.74 | 3 | 444275.8 | 15853.8 | 4 | 537775.8 | 19038 | 4 |
| concentration 2 | 372197.2 | 10208.8 | 3 | 664725.8 | 13697 | 2 | 789838.5 | 10944.7 | 1 |
| concentration 3 | 757557.5 | 40202 | 5 | 1233133 | 59312.9 | 5 | 1427703 | 75575.7 | 5 |

In order to measure in percentage, the component's intensity in the MB and assess if they follow the same reduction as the CB (from 100 to 40 wt %), equation 1 was used:

$$component \% in MB = 100 * \frac{{I}_{M B}}{{I}_{P}} \qquad ( 1 )$$

where
I_{MB} is the average intensity of the 3 components in the MB
Iₚ is the average intensity of the 3 components in the pelletized CB

The average results are shown in Table 13 for the different concentration of marker system A & B. As can be seen, all the concentrations showed on average 40 wt % components loading in the MB which perfectly aligned with the CB loading in the MB. This again supports the suggestion that the components are homogenously dispersed.

TABLE 13
Average components loading the CB MB

| | Marker system A | | | Marker system B | | |
|---|---|---|---|---|---|---|
| | | Average | R. STD | | Average | R. STD |
| | Conc. | (%) | (%) | Conc. | (%) | (%) |
| Component 1 | 1 | 39 | 0.02 | 1 | 41 | 0.03 |
| Component 2 | 2 | 38 | 0.01 | 2 | 41 | 0.03 |
| Component 3 | 3 | 40 | 0.01 | 3 | 41 | 0.03 |

### Samples Production Step

### Dispersion Quality Analysis

The average intensity results and relative STD for all the combinations in thick samples are shown in Table 14 and Table 15 for marker system A and B respectively. As expected, all components showed increase in intensity with increasing CB MB loading. Looking at the relative STD values (=dispersion quality), no clear trend was observed with increasing component concentration. In marker system A, component 1 presented good dispersion, component 2 poor dispersion and component 3 medium dispersion in the final product. In marker system B, component 1 showed inferior dispersion (higher relative STD values) compared to components 2 and 3 in concentrations 1 and 2. In concentration 3, all components showed decrease in dispersion quality.

TABLE 14
Average intensity for all the combinations in Marker system A on thick samples

| | | Marker System A | | | | | | | | |
|---|---|---|---|---|---|---|---|---|---|---|
| | Wt % | Component 1 | | | Component 2 | | | Component 3 | | |
| | marked | Average | STD | R. STD | Average | STD | R. STD | Average | STD | R. STD |
| | CB MB | (a.u) | (a.u) | (%) | (a.u) | (a.u) | (%) | (a.u) | (a.u) | (%) |
| Conc. 1 | 0.5 | 88.19 | 6.22 | 7% | 32.53 | 16 | 49% | 64.68 | 9.6 | 15% |
| | 1 | 118.06 | 5.32 | 5% | 40.84 | 12.18 | 30% | 108.8 | 13.62 | 13% |
| | 2 | 228.03 | 27.35 | 12% | 105.1 | 23.27 | 22% | 208.48 | 38.97 | 19% |
| Conc. 2 | 0.5 | 98.55 | 4.52 | 5% | 26.05 | 10.66 | 41% | 77.56 | 8.9 | 11% |
| | 1 | 155.44 | 17.26 | 11% | 59.11 | 10.42 | 18% | 164.36 | 25.95 | 16% |
| | 2 | 281.23 | 35.91 | 13% | 137.14 | 46.99 | 34% | 261.79 | 21.04 | 8% |
| Conc. 3 | 0.5 | 151.85 | 18.79 | 12% | 55.3 | 11.08 | 20% | 131.43 | 25.04 | 19% |
| | 1 | 279.94 | 46.75 | 17% | 113.66 | 29.03 | 26% | 239.96 | 55.49 | 23% |
| | 2 | 605.53 | 19.22 | 3% | 287.31 | 24.85 | 9% | 574.63 | 22.89 | 4% |

TABLE 15
Average intensity for all the combinations in Marker system B on thick samples

| | | Marker System B | | | | | | | | |
|---|---|---|---|---|---|---|---|---|---|---|
| | wt % | Component 1 | | | Component 2 | | | Component 3 | | |
| | marked | Average | STD | R. STD | Average | STD | R. STD | Average | STD | R. STD |
| | CB MB | (a.u) | (a.u) | (%) | (a.u) | (a.u) | (%) | (a.u) | (a.u) | (%) |
| Conc. 1 | 0.5 | 50.23 | 7.63 | 15% | 105.88 | 6.6 | 6% | 108.9 | 4.69 | 4% |
| | 1 | 109.1 | 9.21 | 8% | 257.35 | 17.57 | 7% | 323.63 | 30.28 | 9% |
| | 2 | 188.47 | 16.45 | 9% | 453.47 | 43.9 | 10% | 574.83 | 62.86 | 11% |
| Conc. 2 | 0.5 | 73.85 | 13.73 | 19% | 193.98 | 14.16 | 7% | 236.09 | 18.74 | 8% |
| | 1 | 97.58 | 13.8 | 14% | 303.46 | 34.98 | 12% | 377.58 | 35.05 | 9% |
| | 2 | 243.35 | 12.3 | 5% | 749.34 | 33.34 | 4% | 972.06 | 33.11 | 3% |
| Conc. 3 | 0.5 | 119.81 | 21.8 | 18% | 352.68 | 70.21 | 20% | 467.27 | 103.14 | 22% |
| | 1 | 280.42 | 38.2 | 14% | 737.15 | 102.56 | 14% | 1026.93 | 165.24 | 16% |
| | 2 | 471.23 | 80.49 | 17% | 1287.5 | 187.99 | 15% | 1798 | 227.01 | 13% |

The average intensity results and relative STD for all the combinations on thin foils was also studied and results are shown in Table 16 and 17 for marker system A and B respectively. For marker system A the analysis was made on 4 foil layers whereas for marker system B on single layer. Same as also shown on thick samples, all components showed increase in intensity with increasing CB MB loading, this was expected as the actual loading of the component increases with increasing CB MB loading. Moreover, with increasing components' concentration no trend was observed in relative STD indicating that the dispersion quality did not change. Same observation given for Marker system A on thick samples is seen on foils where component 1 presented good dispersion, component 2 poor dispersion and component 3 medium dispersion in the final product. In marker system B, component 1 showed inferior dispersion (higher relative STD values) compared to components 2 and 3 in all concentrations. Unlike the thick samples, concentration 3 showed similar dispersion quality to concentration 1 and 2.

TABLE 16
Average intensity for all the combinations in Marker system A on thin foils

| | | Marker System A | | | | | | | | |
|---|---|---|---|---|---|---|---|---|---|---|
| | Wt % | Component 1 | | | Component 2 | | | Component 3 | | |
| | marked | Average | STD | R. STD | Average | STD | R. STD | Average | STD | R. STD |
| | CB MB | (a.u) | (a.u) | (%) | (a.u) | (a.u) | (%) | (a.u) | (a.u) | (%) |
| Conc. 1 | 0.5 | 73.4 | 9.39 | 13% | 21.73 | 12.54 | 58% | 13.49 | 2.04 | 15% |
| | 1 | 81.09 | 9.04 | 11% | 22.53 | 13.14 | 58% | 22.93 | 1.76 | 8% |
| | 2 | 113.47 | 19.58 | 17% | 15.06 | 4.87 | 32% | 43.45 | 6.66 | 15% |
| Conc. 2 | 0.5 | 67.49 | 5.06 | 8% | 18.81 | 8.73 | 46% | 11.91 | 2.15 | 18% |
| | 1 | 92.56 | 12.1 | 13% | 19.32 | 6.13 | 32% | 28.22 | 2.27 | 8% |
| | 2 | 128.22 | 18.62 | 15% | 22.39 | 10.01 | 45% | 53.42 | 6.27 | 12% |
| Conc. 3 | 0.5 | 100.29 | 5.22 | 5% | 22.23 | 11.02 | 50% | 29.84 | 4.57 | 15% |
| | 1 | 148.46 | 17.81 | 12% | 33.97 | 21.07 | 62% | 59.05 | 4.59 | 8% |
| | 2 | 210.84 | 15.39 | 7% | 34.12 | 16.97 | 50% | 97.8 | 9.8 | 10% |

TABLE 17
Average intensity for all the combinations in Marker system B on thin foils

| | | Marker System B | | | | | | | | |
|---|---|---|---|---|---|---|---|---|---|---|
| | Wt % | Component 1 | | | Component 2 | | | Component 3 | | |
| | marked | Average | STD | R. STD | Average | STD | R. STD | Average | STD | R. STD |
| | CB MB | (a.u) | (a.u) | (%) | (a.u) | (a.u) | (%) | (a.u) | (a.u) | (%) |
| Conc. 1 | 0.5 | 6.76 | 1.62 | 24% | 24.31 | 2.38 | 10% | 45.9 | 5.01 | 11% |
| | 1 | 10.78 | 2.72 | 25% | 30.02 | 4.14 | 14% | 56.19 | 5.35 | 10% |
| | 2 | 17.35 | 2.81 | 16% | 44.52 | 4.37 | 10% | 71.61 | 4.52 | 6% |
| Conc. 2 | 0.5 | 7.12 | 1.6 | 22% | 26.85 | 2.7 | 10% | 48.62 | 5.98 | 12% |
| | 1 | 12.07 | 1.61 | 13% | 33.07 | 3.12 | 9% | 60.43 | 2.41 | 4% |
| | 2 | 21.28 | 2.82 | 13% | 54.22 | 3.64 | 7% | 82.36 | 4.45 | 5% |
| Conc. 3 | 0.5 | 11.88 | 1.74 | 15% | 33.91 | 3.49 | 10% | 57.61 | 6.85 | 12% |
| | 1 | 21.92 | 2.14 | 10% | 51.6 | 4.74 | 9% | 82.04 | 5.21 | 6% |
| | 2 | 44.42 | 3.46 | 8% | 97.63 | 7.83 | 8% | 137.38 | 15.05 | 11% |

### Separation Between Marked and Unmarked Products

The aim was to design one marking solution the is capable to distinguish marked from unmarked product for variety of applications that use different CB MB loadings ranging from approx. 0.5 to 2 wt %. Hence, finding the right marker system concentration that is suitable for different CB MB loadings on both thin (foils) and thick (injected) samples was studied.

### Thick Samples (Injected Parts)

The results for thick samples are shown in table 18 and 19 for marker system A and B respectively. The results show that for thick samples, the lowest marker concentration (conc. 1) is sufficient to differentiate marked from unmarked sample in all the different CB MB loadings (0.5, 1 and 2 wt %) with accuracy greater than 95%.

TABLE 18
Minimum components concentrations needed in marker system A to
differentiate marked from unmarked thick sample for different applications

| | Marker system A | | | | | |
|---|---|---|---|---|---|---|
| | Components' conc. needed to differentiate | | | | | |
| | 0.5 wt % CB MB | | 1 wt % CB MB | | 2 wt % CB MB | |
| Injected | loading from | | loading from | | loading from | |
| samples | reference | Accuracy | reference | Accuracy | reference | Accuracy |
| component 1 | Conc. 1 | >95% | Conc. 1 | >95% | Conc. 1 | >95% |
| component 3 | Conc. 1 | >95% | Conc. 1 | >95% | Conc. 1 | >95% |

*Component 2 was excluded from the analysis due to poor performance

TABLE 19
Minimum components concentrations needed in marker system B to
differentiate marked from unmarked thick sample for different applications

| | Marker System B | | | | | |
|---|---|---|---|---|---|---|
| | Components' conc. needed to differentiate | | | | | |
| | 0.5 wt % CB MB | | 1 wt % CB MB | | 2 wt % CB MB | |
| Injected | loading from | | loading from | | loading from | |
| samples | reference | Accuracy | reference | Accuracy | reference | Accuracy |
| component 1 | Conc. 1 | >95% | Conc. 1 | >95% | Conc. 1 | >95% |
| component 2 | Conc. 1 | >95% | Conc. 1 | >95% | Conc. 1 | >95% |
| component 3 | Conc. 1 | >95% | Conc. 1 | >95% | Conc. 1 | >95% |

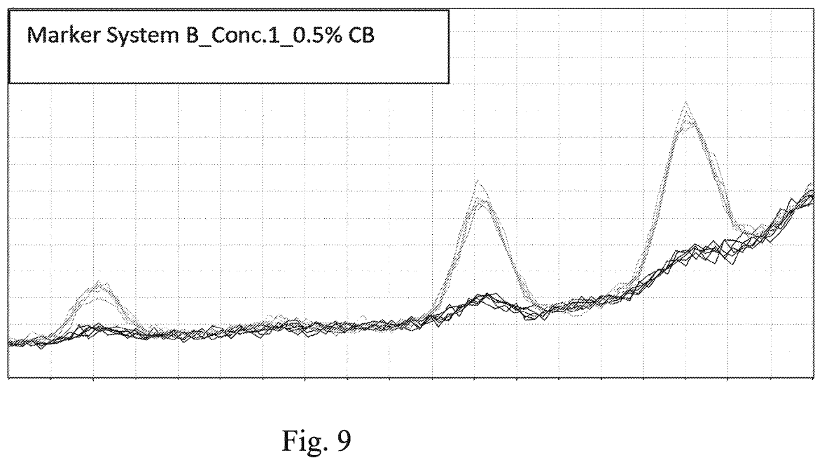

To emphasize high separation capability between marked and unmarked sample, the spectrum of marker system B conc.1 at 0.5 wt % CB MB loading is presented in FIG. 9. The black spectrum represents reference sample (unmarked) while the red spectrum represents the marked sample. As can be clearly seen, all three components present good peak repeatability and don't overlap with the reference line.

### Thin Samples (25 μm Foils)

Same analysis was conducted on thin films and the results for marker system A are presented in Table 20. From 4 layers onwards (>100 μm) good differentiation between marked and unmarked sample is obtained for all the different CB MB loadings (0.5, 1 and 2 wt %) with minimum accuracy of 86%. As expected, at the lowest CB MB loading (0.5 wt %) the maximum marker concentration needed (conc. 3) and with increasing CB MB loading to 1 and 2 wt % the required marker concentration decreases to conc. 2. and conc. 1.

TABLE 20
Minimum components concentrations needed in marker system A to

[symbol]

| | Marker system A | | | | | |
|---|---|---|---|---|---|---|
| | Components' conc. needed to differentiate | | | | | |
| | 0.5 wt % CB MB | | 1 wt % CB MB | | 2 wt % CB MB | |
| Blown film- | loading from | | loading from | | loading from | |
| 4 layers | reference | Accuracy | reference | Accuracy | reference | Accuracy |
| component 1 | Conc. 3 | >95% | Conc. 3 | >95% | Conc. 1 | >86% |
| component 3 | Conc. 3 | >95% | Conc. 2 | >95% | Conc. 2 | >95% |

*Component 2 was excluded due to poor performance [symbol]
indicates data missing or illegible when filed

In marker system B, superior results were obtained. The results in Table 21 show that from 1 layer onwards (>25 μm) good differentiation between marked and unmarked sample is obtained for the different CB MB loadings (0.5, 1 and 2 wt %) with minimum accuracy of 80%. The same trend observed in marker system A follows here where at the minimum CB MB loading (0.5 wt %) high marker concentration (conc.3) is required and with increasing CB MB loading (1 and 2 wt %) the required marker concentration decreases (conc2. And conc. 1).

TABLE 21
Minimum components concentrations needed in marker system B to
differentiate marked from unmarked thick sample for different applications

| | Marker system B | | | | | |
|---|---|---|---|---|---|---|
| | Components' conc. needed to differentiate | | | | | |
| | 0.5 wt % CB MB | | 1 wt % CB MB | | 2 wt % CB MB | |
| Blown film - | loading from | | loading from | | loading from | |
| 1 layer | reference | Accuracy | reference | Accuracy | reference | Accuracy |
| component 1 | Conc. 3 | >95% | Conc. 2 | >95% | Conc. 1 | >95% |
| component 2 | Conc. 2 | > 80% | Conc. 2 | >86% | Conc. 1 | >95% |
| | | | Conc. 3 | >95% | | |
| component 3 | Conc. 3 | >95% | Conc. 2 | >86% | Conc. 1 | >95% |
| | | | Conc. 3 | >95% | | |

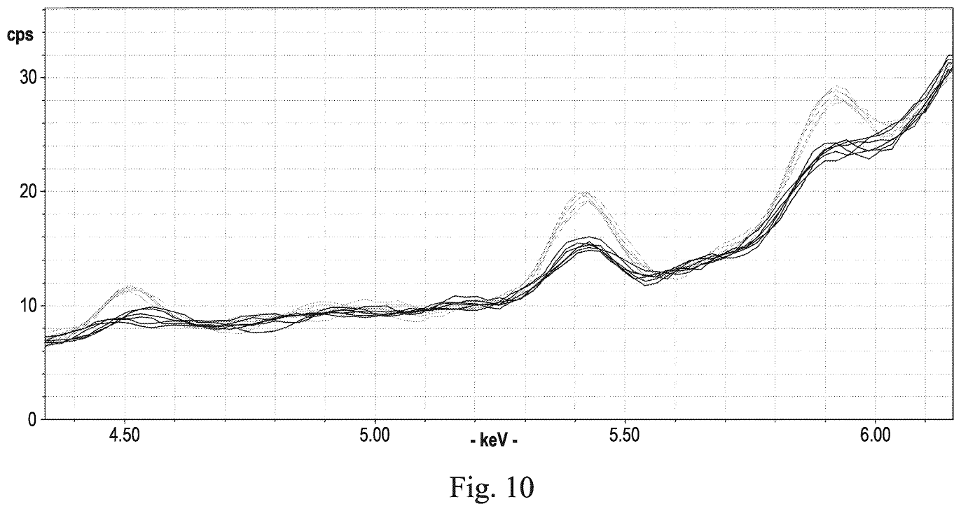

FIG. 10 plots the spectrum for marker system B conc.1 at 2 wt % CB MB loading to show the separation capability between marked and unmarked film. The black spectrum represents reference sample (unmarked) while the blue spectrum represents the marked sample. As can be clearly seen, all three components present good peak repeatability and do not overlap with the reference line.

### Distinction Between the Different CB Loadings

The ability to separate different CB MB loading was also studied with the goal to show the XRF identifiable marker ability to generate multiple codes by using the same components at different concentrations.

The ability of marker system A to separate accurately between ref to 0.5 wt %, 0.5 to 1 wt % and 1 to 2 wt % CB MB loading is presented in Table 22. The results show that at conc.1 all MB concentrations can be separated with accuracy >86%. At conc.2 all MB concentrations can be separated with accuracy >95%. Surprisingly, at conc.3 all MB concentrations can be separated with accuracy >68%. From the intensity results of conc. 3, 1 wt % CB MB loading did not present ×2 increase in intensity from 0.5% MB. Since this was true for all the components, we believe there might be a weighing error at 1 wt % CB loading. It should be noted that based on 9 measurements at different locations, none of the peaks were overlapping with each other between ref, 0.5, 1 and 2, and the accuracy is based on statistics only.

TABLE 22
Separation of different CB MB loading as function
of marker system concentration for thick samples.

| | | % Accuracy between | | |
|---|---|---|---|---|
| Marker System A | | Ref-0.5 wt % | 0.5-1 wt % | 1-2 wt % |
| Injected samples | | MB | MB | MB |
| Component 1 | Conc. 1 | 99.7 | 98 | 99.7 |
| Component 2 | | 99.7 | 86 | 86 |
| Component 1 | Conc. 2 | 99.7 | 95 | 95 |
| Component 2 | | 99.7 | 95 | 95 |
| Component 1 | Conc. 3 | 99.7 | 86 | 99.7 |
| Component 2 | | 99.7 | 68 | 99.7 |

The ability of marker system B to separate accurately between ref to 0.5 wt %, 0.5 to 1 wt % and 1 to 2 wt % CB MB loading is presented in Table 23. At conc.1 all CB MB concentrations can be separated with accuracy >98%. At conc.2 all MB concentrations can be separated with accuracy >86%. Surprisingly, at conc.3, all MB concentrations can be separated with accuracy >68%. This supports our previous observation in section 6.4.1 that all components showed decrease in dispersion quality (=high relative STD) in concertation 3. Same as noted for marker system A, based on 9 measurements none of the peaks were overlapping with each other between ref, 0.5, 1 and 2, and the accuracy is based on statistics only.

TABLE 23
Separation of different CB MB loading as function
of marker system concentration for thick samples.

| | | % Accuracy between | | |
|---|---|---|---|---|
| Marker System B | | Ref-0.5 wt % | 0.5-1 wt % | 1-2 wt % |
| Injected samples | | MB | MB | MB |
| Component 1 | Conc. 1 | 99.7 | 99.7 | 99.7 |
| Component 2 | | 99.7 | 99.7 | 99.7 |
| Component 3 | | 99.7 | 99.7 | 98 |
| Component 1 | Conc. 2 | 99.7 | 95 | 99.7 |
| Component 2 | | 99.7 | 95 | 99.7 |
| Component 3 | | 99.7 | 95 | 99.7 |
| Component 1 | Conc. 3 | 99.7 | 98 | 68 |
| Component 2 | | 99.7 | 95 | 86 |
| Component 3 | | 99.7 | 99.7 | 86 |

### Thin Samples (25 μm Foils)

The ability of marker system A to separate accurately between ref to 0.5 wt %, to 1 wt % and 1 to 2 wt % CB MB loading on 4 layers of foils is presented in Table 24. As can be seen from the table below, at conc.3 all MB concentrations can be separated with minimum accuracy of 86%. Component 1 showed increase in accuracy with increasing its concentration, while component 2 showed accuracy of 99.7% in all the concentration. It should be noted that based on 9 measurements at different locations, none of the peaks were overlapping with each other between ref, 0.5, 1 and 2, and the accuracy is based on statistics only.

TABLE 24
Separation of different CB MB loading as function
of marker system concentration for thin samples.

| | | % Accuracy between | | |
|---|---|---|---|---|
| Marker System A | | Ref-0.5 wt % | 0.5-1 wt % | 1-2 wt % |
| Thin film - 4 layers | | MB | MB | MB |
| Component 1 | Conc. 1 | 38 | 0 | 68 |
| Component 2 | | 99.7 | 99.7 | 99.7 |
| Component 1 | Conc. 2 | 38 | 68 | 68 |
| Component 2 | | 99.7 | 99.7 | 99.7 |
| Component 1 | Conc. 3 | 99.7 | 95 | 86 |
| Component 2 | | 99.7 | 99.7 | 99.7 |

The ability of marker system B to separate accurately between ref to 0.5 wt %, to 1 wt % and 1 to 2 wt % CB MB loading on single foil layer is presented in Table Marker system B presents superior results one single foil layer and at conc.3 all MB concentrations can be separated with minimum accuracy of 95%. All components showed increase in accuracy with increasing their concentration. Same as noted for marker system A, based on 9 measurements none of the peaks were overlapping with each other between ref, 0.5, 1 and 2, and the accuracy is based on statistics only.

TABLE 25
Separation of different CB MB loading as function
of marker system concentration for thin samples.

| | | % Accuracy between | | |
|---|---|---|---|---|
| Marker System B | | Ref-0.5 wt % | 0.5-1 wt % | 1-2 wt % |
| Thin film - 1 layer | | MB | MB | MB |
| Component 1 | Conc. 1 | 38 | 38 | 68 |
| Component 2 | | 0 | 38 | 86 |
| Component 3 | | 38 | 38 | 86 |
| Component 1 | Conc. 2 | 68 | 86 | 95 |
| Component 2 | | 38 | 68 | 99.7 |
| Component 3 | | 38 | 68 | 99.7 |
| Component 1 | Conc. 3 | 99.7 | 98 | 99.7 |
| Component 2 | | 95 | 95 | 99.7 |
| Component 3 | | 95 | 95 | 95 |

1-26. (canceled)

27. A composition comprising carbon black and at least one XRF-identifiable material, the composition being a pigment formulation or a reinforcement formulation, wherein the at least one XRF-identifiable material is present in an amount selected to provide an XRF-identifiable signature indicative of the carbon black or the composition comprising same.

28. The composition according to claim 27, comprising a polymer or a prepolymer.

29. An XRF-identifiable masterbatch composition comprising a homogenous blend of carbon black, at least one XRF-identifiable marker and at least one polymer or prepolymer.

30. The composition according to claim 28, wherein the polymer is a thermoplastic polymer or thermoset polymer.

31. The composition according to claim 30, wherein the polymer is selected from Low-Density Polyethylene (LDPE), Linear Low-Density Polyethylene (LLDPE), High-Density Polyethylene (HDPE), Polypropylene (PP), Polyisoprenes, natural rubber and latex.

32. The composition according to claim 27, wherein the ratio between carbon black and the at least one XRF-identifiable marker is at least 100:1, respectively.

33. A method for providing an XRF-identifiable black polymeric raw material, the method comprising marking a polymeric raw material with an amount of an XRF-identifiable marker and black carbon, the amount of the XRF-identifiable marker defining an electromagnetic radiation signature indicative of the raw material composition and/or production profile (a raw material data).

34. The method according to claim 33, wherein the profile comprises one or more data of manufacture, site of manufacture, composition, and presence or absence of unnatural additives.

35. A method for identifying a black plastic during sorting of plastic materials, the method comprising:
irradiating with X-Ray or Gamma-Ray radiation a collection of plastic objects comprising black objects marked with at least one XRF-identifiable marker;
detecting an X-Ray or Gamma-Ray signal arriving from the objects in response to the X-Ray or Gamma-Ray radiation applied thereto; and
applying spectral processing to the detected radiation signal to obtain data indicative of the presence, absence or any change in the predefined characteristic relating to the black plastic.

36. The method according to claim 35, the method comprising: simultaneously irradiating a plurality of objects with at least one X-ray or Gamma-ray excitation beam having a spatially distributed modulated intensity; wherein the intensity of the beam arriving at each of the objects is different and identifiable and wherein the plurality of objects comprising black objects;
detecting a secondary X-ray radiation arriving from the plurality of objects and generating signals indicative of the spatial intensity distribution on the plurality of objects; and
identifying which of the plurality of black objects are marked by a marking composition according to the detected spatial intensity distribution.

37. The method according to claim 35, wherein the black objects are formed by marking a black plastic with at least one XRF-identifiable marker.

38. The method according to claim 35, wherein the predefined identifiable characteristic comprises the XRF-identifiable pattern concentration or encryption code.

39. A method of sorting black objects in a recycling process, the method comprising:
providing measured data indicative of an electromagnetic radiation signature embedded in a black object;
identifying radiation emitted from a material in response to X-Ray or gamma-ray radiation, said radiation having spectral features characteristic of the signature, thereby determining whether the material is a black object.

40. An X-Ray Fluorescence (XRF) method of managing black material recycling process, the method comprising:
providing first measured data indicative of one or more first electromagnetic radiation signatures embedded in one or more black plastic object;
analyzing the measured data to determine, for each of said one or more black plastic object, a respective plastic material condition data, wherein the respective plastic object condition data is indicative of preceding use of said plastic object;
generating first sorting data for each of said one or more black plastic objects, based on the respective plastic material condition; and
generating marking data for at least one of said one or more black plastic objects, based on the first sorting data, wherein the marking data includes data indicative of at least one marker to be introduced into each of said one or more plastic objects to provide electromagnetic radiation signal for managing a recycling process said one or more black plastic object,
wherein the electromagnetic radiation signals of the measured data comprise X-Ray Fluorescence (XRF) signals; and the data indicative of the at least one marker correspond to the at least one marker responding by XRF response signals to XRF exciting radiation.

41. The method according to claim 40, further comprises utilizing at least one of the black plastic objects condition data and the sorting data of said plastic object and generating and storing certificate data characterizing a current condition of said black plastic object to be sorted.

42. The method according to claim 41, wherein the data indicative of the at least one marker is obtained from a database, storing, for each plastic material reuse type, data indicative of a life cycle of said plastic object in association with matching data about corresponding one or more markers.

43. The method according to claim 41, wherein the data indicative of the at least one marker may comprise data corresponding to (a) a number of a successive life cycle of said plastic material being recycled and (b) a successive product type for reuse of recycled plastic object.

44. The method according to claim 41, the method further comprises providing second measured data indicative of one or more second electromagnetic radiation signals originated by one or more contaminant elements presented in the plastic object after being sorted by introducing said marking therein.

45. The method according to claim 41, the method further comprises providing second measured data indicative of one or more second electromagnetic radiation signals originated by one or more contaminant elements presented in the black plastic object after being sorted by introducing said marking therein and updating the certificate data characterizing the black plastic object.

46. An XRF-identifiable pelletized powder comprising a homogenous blend of carbon black and an amount of at least one XRF-identifiable marker.
